(19) United States
(12) Patent Application Publication
(10) Pub. No.: US 2025/0160753 A1
(43) Pub. Date: May 22, 2025

(54) DEVICES, INSERTION APPARATUS, AND METHODS FOR ADMINISTERING SAMPLES

(71) Applicants: Abhinav GOYAL, Haryana (IN); Vikas Khurana, South Abington Township, PA (US)

(72) Inventors: Abhinav GOYAL, Haryana (IN); Vikas Khurana, South Abington Township, PA (US)

(21) Appl. No.: 18/834,720

(22) PCT Filed: Feb. 1, 2023
(86) PCT No.: PCT/IB2023/050863

§ 371 Date: Jul. 31, 2024

(30) Foreign Application Priority Data
Feb. 1, 2022 (IN) 202211005464

(51) Int. Cl. A61B 5/00

(52) U.S. Cl. CPC A61B 5/6861; A61B 5/6873; A61B 2562/162

(57) ABSTRACT

Disclosed is one or more airbags (106A-106N), each airbag of the one or more airbags (106A-106N) is positioned on the capsular device (100), adapted to inflate such that one or more airbags (106A-106N) locks the capsular device (100) in a point of interest in a gastrointestinal tract and prevents the capsular device (100) to move from the point of interest, and one or more needles (108A-108N) are positioned in the shell (104), each needle of the one or more needles (108A-108N) is adapted to move in a substantially horizontal direction and adapted to inject at least one sample of one or more samples to the point of interest on the gastrointestinal tract. The present disclosure also relates to a system and method for administering a capsular device (100).

### TECHNICAL FIELD

The present disclosure relates generally to a medical device and more particularly to the device and methods for testing immunological response of a subject.

### BACKGROUND

More than 50 million Americans have an allergy of some kind. Food allergies are estimated to affect 4%-6% of children and 4% of adults, according to the Centers for Disease Control and Prevention. Food allergy symptoms are most common in babies and children, but it can appear at any age.

Symptoms of a food allergy can range from mild to severe. Just because an initial reaction causes few problems doesn't mean that all reactions will be similar; a food that triggered only mild symptoms on one occasion may cause more severe symptoms at another time.

Eosinophilic esophagitis (EoE) is an allergic condition that happens in the esophagus. During EoE, the esophagus becomes inflamed and does not contract properly. Also, the esophagus gets narrow and forms strictures due to scarring from inflammation. This happens as a result of eosinophils (a type of white blood cell) being attracted to the esophagus in reaction to an allergen.

The main cause of EoE is thought to be an allergic reaction to certain foods or environmental allergens.

Generally, EoE is diagnosed by endoscopy and biopsies obtained from esophagus.

The skin-based allergen testing is used to find out various foods and environmental allergens that the patient is allergic to and then eliminate those from the diet as a form of treatment for eosinophilic esophagitis.

However, this has not been very successful due to poor response rates. This is likely due to poor correlation with skin testing. More recently empiric elimination diets have been used to manage EoE. However, these diets can be very restrictive with questionable long-term compliance from patients as EoE is a chronic disease without a cure. This non-compliance eventually leads to unchecked inflammation through an incompletely understood pathway which as a result involves eosinophils homing to the esophageal mucosa and releasing local inflammatory mediators that result in dysphagia and strictures.

The endoscopy-based testing is an outpatient procedure that involves passing a thin, flexible endoscope with a camera through the mouth and down the throat to look at the esophagus. The gastroenterologist will check for physical signs of inflammation and obtain biopsies to look for an increased number of eosinophils.

Thus, there is a need for the development of technology to understand allergic reactions in the esophagus of a person to advise personalized diet based on the results associated with the allergic reaction.

### SUMMARY

In one aspect of the present disclosure, a capsular device is provided.

The capsular device includes one or more airbags, each airbag of the one or more airbags is positioned on both proximal and distal sides of the capsular device, adapted to inflate and locks the capsular device in a point of interest in a gastrointestinal tract and prevents the capsular device to move from the point of interest. The capsular device further includes one or more needles that are positioned in the capsule device, each needle of the one or more needles is adapted to move in a substantially horizontal direction and adapted to inject at least one sample of one or more samples to the point of interest on the gastrointestinal tract.

The capsular device further includes a tube, such that the tube facilitates a clinician to pump a pumping agent into each airbag of the one or more airbags.

In some aspects of the present disclosure, the tube facilitates the clinician to dispense each sample of one or more samples to the points of interest.

In some aspects of the present disclosure, each needle of the one or more needles comprising at least one payload of the one or more payloads, each payload of the one or more payloads is adapted to store at least one sample, upon injection, each payload of the one or more payloads is adapted to provide a corresponding sample of the one or more samples to a corresponding needle of the one or more needles.

In some aspects of the present disclosure, the capsular device further includes a control unit that is positioned inside the capsular device, adapted to control a flow of samples to the respective needle of the one or more needles.

In some aspects of the present disclosure, the control unit further includes a Radio Frequency Identity Card (RFID), that facilitates the clinician to locate the capsular device in the subject.

In some aspects of the present disclosure, the control unit further includes one or more sensors, that are configured to detect one or more parameters associated with the subject.

In some aspects of the present disclosure, a capsular device is disclosed. The capsular device includes one or more airbags, each airbag of the one or more airbags is positioned on both proximal and distal sides of the capsular device, and adapted to inflate and lock the capsular device in a point of interest in a gastrointestinal tract and prevents the capsular device to move from the point of interest. The capsular device further includes one or more needles that are positioned in the capsule device, each needle of the one or more needles is adapted to move in a substantially horizontal direction and adapted to inject at least one allergen of one or more allergens to the point of interest on the gastrointestinal tract.

In some aspects of the present disclosure, each needle of the one or more needles includes at least one payload of one or more payloads such that the at least one payload of the one or more payloads stores (i) the at least one allergen and (ii) one or more agents. The one or more agents is one of, food grade dyes and coloring agents. The at least one allergen is mixed with the one or more agents in the at least one payload of the one or more payloads. The addition of the one or more agents may facilitate visual inspection of the site after injection of the one or more allergens to the site to confirm accurate placement.

In some aspects of the present disclosure, the point of interest is mucosa in the gastrointestinal tract.

In another aspect of the present disclosure, a pellet device is provided.

The pellet device includes a housing, that is adapted to encapsulate the pellet device. The pellet device further includes a pair of locking bags, at least one locking bag of the pair of locking bags is positioned on proximal and distal end of the pellet device, adapted to lock the pellet device in a region of interest in a gastrointestinal tract, and prevent the pellet device to move from the region of interest. The pellet device further includes one or more cameras, each camera of the one or more cameras is positioned on the pellet device, facilitates a clinician to visualize walls of the gastrointestinal tract, such that the clinician identifies the region of interest. The pellet device further includes a suction pump, that is adapted to create vacuum in a region between the region of interest and the pellet device. The pellet device further includes one or more pins, each pin of the one or more pins is adapted to move in a substantially horizontal direction and inject at least one sample of one or more samples to the region of interest.

In some aspects of the present disclosure, the pellet device further includes a hose, that is positioned on the pellet device and facilitates the clinician to operate the pellet device.

In some aspects of the present disclosure, the pellet device further includes one or more detectors, that are adapted to detect one or more parameters.

In another aspect of the present disclosure, an installing apparatus is provided.

The installing apparatus includes a core that is adapted to be administered along a gastrointestinal tract of a subject. The installing apparatus includes an inflation equipment that includes a pipe that is operatively positioned inside the core of the insertion apparatus. The installing apparatus further includes a bladder that is coupled with the pipe, the bladder includes an expandable layer that is adapted to inflate, upon receiving a pumping agent from the pipe. The bladder further includes one or more nails, each nail of the one or more nails is mounted over the expandable layer, and upon inflating, each nail of the one or more nails is adapted to inject one or more samples into a region of interest of a subject. The bladder further includes one or more compartments, at least one compartment of the one or more compartments is positioned on proximal end of each nail of the one or more nails. Each compartment of the one or more compartments is adapted to store at least one sample of the one or more samples, upon inflating the pumping agent squeezes each compart of the one or more compartments. Such that at least one sample of the one or more samples stored in each compartment of the one or more compartments is injected to the region of interest of the subject by way of the one or more nails.

In another aspect of the present disclosure, an implanting apparatus is provided.

The implanting apparatus includes a delivery device, that is adapted to be administered along a gastrointestinal tract of a subject;

The implanting apparatus further includes an inflation equipment that is operatively positioned inside the delivery device of the implanting apparatus. The inflation equipment includes a bag with one or more cavities that are positioned on the bladder and adapted to project externally upon inflation. The bag includes one or more chambers, at least one chamber is positioned on each cavity of the one or more cavities.

Each chamber of the one or more chambers is adapted to store at least one sample of one or more samples. The bag includes one or more infusers, at least one infuser is positioned on each chamber of the one or more chambers, adapted to transmit corresponding sample of the one or more samples stored in a respective chamber of the one or more chambers. Each infuser of the one or more infusers a hook, that is positioned on a distal end of each infuser of the one or more infusers and facilitates the one or more infusers to bind with a region of interest of the gastrointestinal tract.

In another aspect of the present disclosure, a method of injecting one or more samples to a point of interest of a subject is provided.

The method includes administering a capsular device to a subject by orally swallowing the capsular device. The method further includes allowing, upon swallowing, the capsular device to reach a point of interest. The method further includes administering, upon reaching the point of interest, an edible reagent to the subject, such that the edible reagent reacts with the shell of the capsular device, and upon reaction, the edible reagent is adapted to dissolve the shell of the capsular device. The method further includes pumping, upon dissolving the capsular device, air by way of a tube connected with the capsular device, such that one or more airbags inflate and lock the capsular device to moving apart from the point of interest. The method further includes injecting, upon locking, the one or more samples into the point of interest. The method further includes deflating, upon injecting, the one or more airbags such that the capsular unlocks from the point of interest and is further adapted to move from the point of interest.

In another aspect of the present disclosure, a method of injecting one or more samples into one or more regions of interest of the subject.

The method includes administering capsular device to a subject by orally swallowing the capsular device. The method further includes allowing, upon swallowing, the capsular device to reach a first region of the one or more regions of interest. The method further includes administering, upon reaching the first region of interest, an edible reagent to the subject, such that the edible reagent reacts with the shell of the capsular device, and upon reaction, the edible reagent is adapted to dissolve the shell of the capsular device. The method further includes pumping, upon dissolving the capsular device, air by way of a tube connected with the capsular device, such that one or more airbags inflate and lock the capsular device to move apart from the point of interest.

The method further includes injecting, upon locking, a first sample of the one or more samples into the first region of interest. The method further includes deflating, upon injecting, the one or more airbags such that the capsular device unlocks from the first region of interest. The method further includes displacing, upon unlocking, the capsular device from the first region of interest and moving the capsular device to a second region of the one or more regions of interest. The method further includes inflating, upon displacing, one or more airbags to lock the capsular device on a second region of interest of the one or more regions of interest. The method further includes injecting, upon locking, a second sample of the one or more samples into the second region of interest. The method further includes deflating, upon injecting, the one or more airbags such that the capsular device unlocks from the second region of interest and is further adapted to move from the second region of interest.

In another aspect of the present disclosure, a method of administering a capsular device to a point of interest of a subject by way of an insertion equipment. The method includes deploying the capsular device into a docking region of the insertion equipment. The method further includes inserting, upon deploying, the insertion equipment into a gastrointestinal tract of the subject. The method further includes allowing, upon inserting, the insertion equipment to reach a point of interest. The method further includes deploying, upon reaching the point of interest, the capsular device on the point of interest. The method further includes removing, upon deploying, the insertion apparatus from the gastrointestinal tract of the subject. The method further includes activating, upon removing, the capsular device to inject each sample of one or more samples into the point of interest of the subject.

### BRIEF DESCRIPTION OF DRAWINGS

The above and still further features and advantages of aspects of the present disclosure become apparent upon consideration of the following detailed description of aspects thereof, especially when taken in conjunction with the accompanying drawings, and wherein:

FIGS. 1A and 1B illustrates a capsular device, in accordance with an aspect of the present disclosure;

FIG. 2 illustrates an exemplary capsular device, in accordance with an aspect of the present disclosure;

FIG. 3 illustrates an exemplary capsular device that is administered in a gastrointestinal tract of the subject, in accordance with an aspect of the present disclosure;

FIG. 4 illustrates an insertion apparatus, in accordance with an aspect of the present disclosure;

FIGS. 5A, 5B, and 5C illustrate an insertion apparatus, in accordance with an aspect of the present disclosure;

FIGS. 6A and 6B illustrate an insertion apparatus, in accordance with an aspect of the present disclosure;

FIG. 7 illustrates a flowchart that depicts a method of administering one or more samples to the point of interest of the subject, in accordance with an aspect of the present disclosure;

FIG. 8 illustrates a flowchart that depicts a method of administering one or more samples to one or more regions of interest of the subject, in accordance with an aspect of the present disclosure; and

FIG. 9 illustrates a flowchart that depicts a method of administering the capsular device to a point of interest of the subject by way of the insertion equipment, in accordance with an aspect of the present disclosure.

To facilitate understanding, like reference numerals have been used, where possible, to designate like elements common to the figures.

### DETAILED DESCRIPTION

Various aspects of the present disclosure provide a device and methods for testing immunological response of a subject. The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

The various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

Spatially relative terms, such as “top,” “bottom,” and the like, may be used herein for ease of description to describe one element or feature's relationship to another element(s) or feature(s) as illustrated in the figures. It is to be understood that the spatially relative terms are intended to encompass different orientations of the structure in use or operation in addition to the orientation depicted in the figures.

The subject matter of example aspects, as disclosed herein, is described with specificity to meet statutory requirements. However, the description itself is not intended to limit the scope of this patent. Rather, the inventor/inventors have contemplated that the claimed subject matter might also be embodied in other ways, to include different features or combinations of features similar to the ones described in this document, in conjunction with other technologies. Generally, the various aspects including the example aspects relate to the device and method of injecting one or more allergen samples into the esophagus of the subject.

As mentioned, there remains a need for a device and method to understand allergic reactions of the esophagus of a subject.

The present disclosure, therefore, provides a device that facilitates the clinician to inject one or more samples into the esophagus of the subject. The present disclosure also provides a capsular device that facilitates the clinician to inject one or more samples on multiple sites of the esophagus of the subject. The present disclosure also provides insertion apparatus that facilitates the capsular device to reach a point or region of interest.

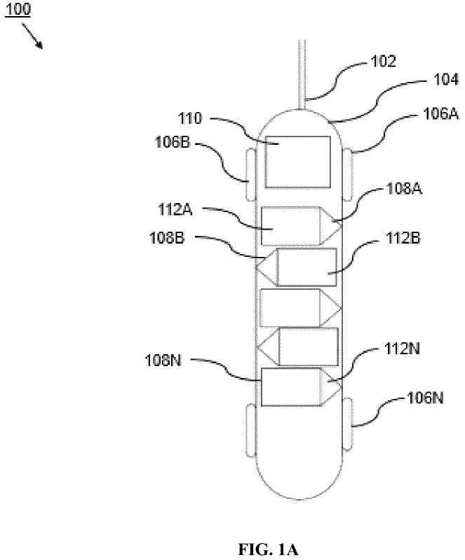

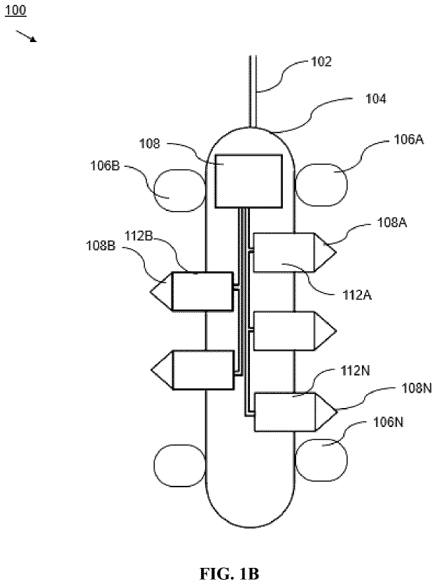

FIGS. 1A and 1B illustrates a capsular device 100, in accordance with an aspect of the present disclosure.

The capsular device 100 may include a tube 102, a shell 104, one or more airbags 106A-106N, one or more needles 108A-108N, and a control unit 110.

The tube 102 may be coupled with the capsular device 100. The tube 102 may facilitate a clinician to pump a pumping agent into each airbag 106A-106N of the one or more airbags 106A-106N. In some aspects of the present disclosure, the tube 102 may facilitate the clinician to dispense each sample of one or more samples to a point of interest. In some aspects of the present disclosure, the one or more samples may be mixed with a food-grade dye. In some other aspects of the present disclosure, the food grade dye may facilitate the clinician to assist with localization of each sample of the one or more samples.

The shell 104 may be positioned over the capsular device 100. The shell 104 may be adapted to encapsulate the capsular device 100. In some aspects of the present disclosure, the shell 104 may be a soluble material. In some aspects of the present disclosure, the shell 104 may be soluble to one or more edible solutions when administered.

The one or more airbags 106A-106N may be positioned on the capsular device 100. In some aspects of the present disclosure, each airbag of the one or more airbags 106A-106N may be positioned in a proximal side of the capsular device 100. In some aspects of the present disclosure, each airbag of the one or more airbags 106A-106N may be positioned in a distal side of the capsular device. In some other aspects of the present disclosure, each airbag of the one or more airbags 106A-106N may be positioned in both proximal and distal sides of the capsular device (for purposes of this disclosure, “distal side” refers to the portion of the capsular device 100 that is far from the tube 102 during normal use and “proximal side” refers to the portion of the capsular device 100 that is closer to the tube 102 during normal use).

Each airbag of the one or more airbags 106A-106N may be positioned in proximal and distal sides of the capsular device 100. The one or more airbags 106A-106N may be adapted to inflate and lock the capsular device 100 in the point of interest. Such that one or more airbags 106A-106N prevents the capsular device 100 to move away from the point of interest. In some aspects of the present disclosure, pumping agent may be pumped into each airbag of the one or more airbags 106A-106N to inflate each airbag of the one or more airbags 106A-106N (as shown in FIG. 1B). In some aspects of the present disclosure, the pumping agent may be pumped into the one or more airbags 106A-106N through the tube 102. In some aspects of the present disclosure, the one or more airbags 106A-106N may be positioned in parallel to each other. In some aspects of the present disclosure, the one or more airbags 106A-106N may be positioned in a periphery of proximal and distal sides of the capsule device 100.

In some other aspects of the present disclosure, the pumping agent may be pumped by a clinician operating the capsular device 100. In some aspects of the present disclosure, the pumping agent may include, but not limited to, air, liquid, gel, and the like.

The one or more needles 108A-108N may be positioned in the capsular device 100. Each needle of the one or more needles 108A-108N may be adapted to move in a substantially horizontal direction 100. In some aspects of the present disclosure, each needle of the one or more needles 108A-108N may be adapted to move in a substantially vertical direction. Upon dissolving the shell 102, each needle of the one or more needles 108A-108N may be adapted to inject at least one sample of the one or more samples to the point of interest. In some aspects of the present disclosure, the point of interest may be a targeted region in the esophagus where the one or more samples may be injected.

In some aspects of the present disclosure, each needle of the one or more needles 108A-108N may be adapted to inject at least one sample of the one or more samples into a point of interest in the oesophagus.

In some aspects of the present disclosure, each needle of the one or more needles 108A-108N comprising at least one payload of the one or more payloads 112A-112N, each payload of the one or more payloads 112A-112N is adapted to store at least one sample of the one or more samples, upon injection, each payload of the one or more payloads 112A-112N is adapted to provide a corresponding sample of the one or more samples to a corresponding needle of the one or more needles 108A-108N.

In some aspects of the present disclosure, the one or more samples may be injected externally by the clinician by way of the tube 102.

The control unit 110 may be positioned in the capsular device 100.

The control unit 110 may be adapted to control flow of each sample of the one or more samples to the respective needle of the one or more needles 108A-108N.

The control unit 110 may further include one or more sensors, that are configured to detect one or more body parameters associated with the subject.

In some aspects of the present disclosure, the capsular device 100 may further include a Radio Frequency Identity Card (RFID), that facilitates the clinician to locate the capsular device 100 in the subject. In some aspects of the present disclosure, the capsular device 100 may be provided with a camera such that the RFID and the camera may together facilitate the clinician to locate the capsular device 100 in the subject. In some aspects of the present disclosure, the RFID may be a passive RFID. In some other aspects of the present disclosure, the RFID may be an active RFID.

In some aspects of the present disclosure, one or more sensors may be one of, a temperature sensor, power of Hydrogen (pH) sensor, breath monitor, infection detector, bleeding detector, microorganism detector, imaging sensor, or texture detector.

In some aspects of the present disclosure, the one or more parameters are one of, temperature, power of Hydrogen (pH), breath level, microbial infection, epithelium, or bleeding.

In some aspects of the present disclosure, the capsular device 100 may facilitate dispensing of one or more allergens into mucosa that may be triggers of eosinophilic esophagitis at different sites in the mucosa of the esophagus. Specifically, the one or more allergens may be stored in the one or more payloads 112A-112N such that the one or more allergens dispense into mucosa by way of the one or more needles 108A-108N. In some examples of the present disclosure, the one or more payloads 112A-112N may be further adapted to store one or more agents such that the one or more agents may be one of, food grade dyes and coloring agents. The one or more agents may therefore be mixed with the one or more allergens in the at least one payload of the one or more payloads 112A-112N. The one or more agents may be mixed with the one or more allergens in the one or more payloads 112A-112N. The one or more needles 108A-108N may be adapted to dispense the one or more agents mixed with the one or more allergens that may advantageously facilitate to accurately identify allergy to the one or more agents while reading results. In other words, the addition of the one or more agents may facilitate visual inspection of the site after injection of the one or more allergens to the site to confirm accurate placement. The one or more airbags 106A-106N may facilitate to secure the capsular device 100 intact into the mucosa. The capsular device 100 may be configured to dispense the one or more allergens at different locations of the mucosa. Specifically, the one or more needles 108A-108N may be configured to dispense the one or more allergens at different locations/sites of the mucosa. The one or more needles 108A-108N may further be configured to dispense the one or more allergens in different patterns.

In some aspects of the present disclosure, the capsular device 100 may facilitate injecting/dispensing the one or more allergens into other parts/sites of the gastrointestinal (GI) tract to assess allergic response/results. Specifically, the one or more allergens may be stored in the one or more payloads 112A-112N such that the one or more allergens dispense into other parts/sites of the GI tract by way of the one or more needles 108A-108N. The results may be assessed at one or more intervals such as Dec. 24, 1948/72 hours after injection/dispensing of the one or more allergens. The results may be assessed by way of a camera-based device or an endoscope.

In some aspects of the present disclosure, the capsular device 100 may facilitate to inject/dispense the one or more allergens along with suitable agents. The suitable agents may dampen response, such as, steroid or antihistamine, and the like.

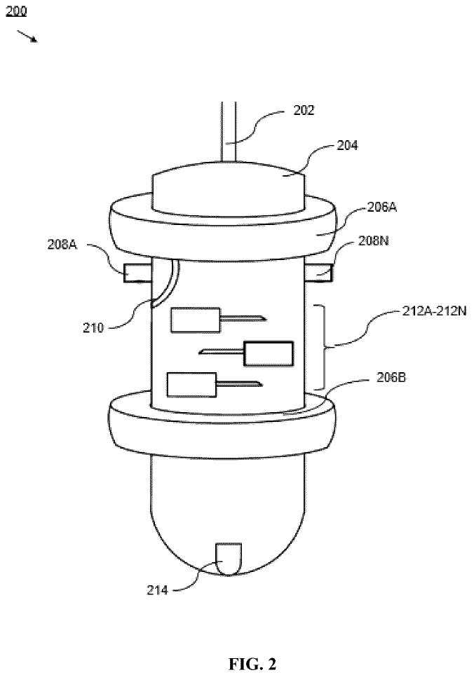

In some aspects of the present disclosure, the capsular device 100 may facilitate injection/dispensing of the one or more allergens/medical agents through endoscopic injection. FIG. 2 illustrates an exemplary pellet device 200, in accordance with an aspect of the present disclosure. The pellet device 200 is substantially similar to that of the capsular device 100 of FIG. 1. However, suction tube 210, one or more locking bags 206A and 206B, and one or more cameras 208A and 208B of the pellet device 200 are different when compared to the capsular device 100 of FIG. 1.

The pellet device 200 may include a hose 202, housing 204, a pair of locking bags 206A-206B, one or more cameras 208A-208N, a suction pump 210, and one or more pins 212A-212N. The utilities of the one or more pins 212A-212N are substantially similar to the utilities of the one or more needles 108A-108N of capsular device 100. In order to differentiate the components with respect to Figures alike terms are used.

The hose 202 may be coupled with the housing 204 on the pellet device 200. The hose 202 may facilitate the clinician to operate the pellet device 200. The hose 202 may facilitate the clinician to inflate each locking bag of the pair of locking bags 206A-206B.

The housing 204 may be adapted to encapsulate the pellet device 200.

A first locking bag of the pair of locking bags 206A-206B may be positioned on a proximal side of the capsular device 100. A second locking bag of the pair of locking bags 206A-206B may be positioned on a distal side of the pellet device 200 (for purposes of this disclosure, “distal side” refers to the portion of the pellet device 200 that is far from the hose 202 during normal use and “proximal side” refers to the portion of the pellet device 200 that is closer to the hose 202 during normal use).

Each locking bag of the pair of locking bags 206A-206B may be adapted to inflate, upon receiving the inflating agent provided by the clinician. The pair of locking bags 206A-206B may be adapted to lock the capsule in a region of interest 304 (explained in a later part of the description) in a gastrointestinal tract 302 (explained in a later part of the description). The region of interest 304 is substantially similar to the point of interest of FIG. 1. In order to differentiate the components with respect to Figures alike terms are used.

Each camera of the one or more cameras 208A-208N may be positioned on the shell 204. Each camera of the one or more cameras 208A-208N may facilitate the clinician to visualize the walls of the gastrointestinal tract 302. Such that the clinician identifies the region of interest 306 in the walls of the gastrointestinal tract 302.

The suction pump 210 may be positioned in the pellet device 200. Suction side of the suction pump 210 may be adapted to create a vacuum around the pellet device 200.

One or more pins 212A-212N may be positioned in the pellet device 200. Each pin of the one or more pins 212A-212N may be adapted to move in a substantially horizontal direction. Each pin of the one or more pins 212A-212N may be adapted to inject at least one sample of the one or more samples to the region of interest 304.

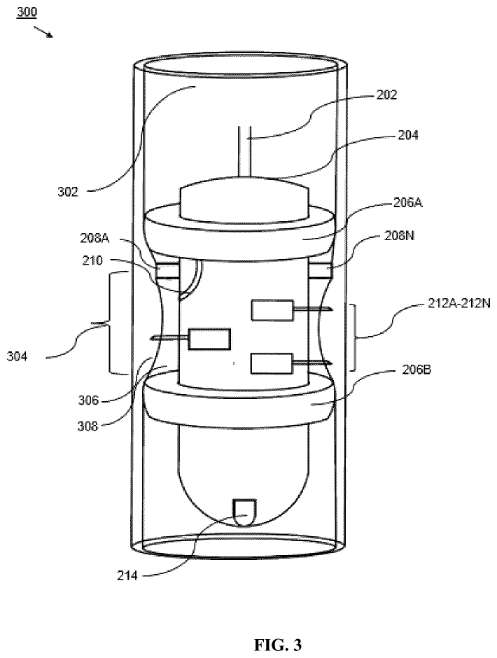

FIG. 3 illustrates an exemplary pellet device 200 that is administered in the gastrointestinal tract 302 of the subject, in accordance with an aspect of the present disclosure.

In an exemplary scenario, the pellet device 200 may be administered in the gastrointestinal tract 302 of the subject. In some aspects of the present disclosure, the pellet device 200 may be administered in the gastrointestinal tract 302 by the clinician. In some other aspects of the present disclosure, the one or more cameras 208A-208N may be adapted to facilitate the clinician to visualize the gastrointestinal tract 302 of the user, such that the clinician identifies the region of interest 304 in the gastrointestinal tract 302. Upon identifying the region of interest 304, the clinician may pump the pumping agent to each locking bag of the one or more locking bags 206A-206N to inflate each bag of the one or more locking bags 206A-206N. Upon inflation, the one or more locking bags may be adapted to lock the capsular device 100 and prevents the capsular device 100 to move from the region of interest. The suction device 210 may be adapted to create a vacuum between the region of interest 304 and the pellet device 200 as shown in FIG. 3 such that the walls of the region of interest 304 are pulled towards the pellet device 200. Upon creating vacuum, the clinician may inject the one or more samples to the region of interest 304 by way of the one or more pins 212A-212N.

In some aspects of the present disclosure, the clinician may pump the one or more samples to the region of interest externally.

In some aspects of the present disclosure, the clinician may inject the one or more samples to the region of interest 304 by storing the one or more samples in payload (not shown) in the pellet device 200.

In some aspects of the present disclosure, each pin of the one or more pins 212A-212N may be nano-encapsulated with at least one sample of the one or more samples.

The pellet device 200 may further include one or more detectors 214, that are adapted to detect one or more parameters representing body condition of the subject.

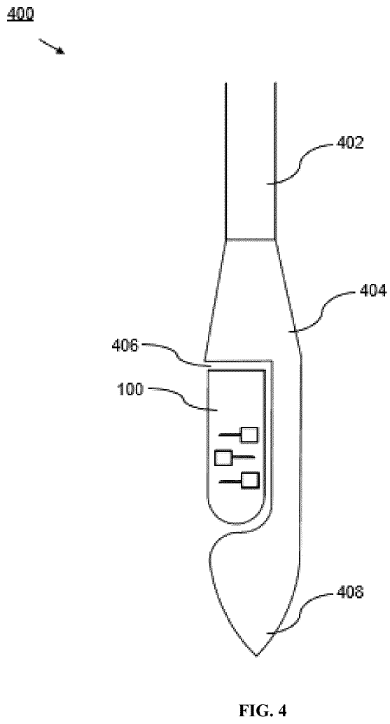

FIG. 4 illustrates an insertion apparatus 400, in accordance with an aspect of the present disclosure.

The insertion apparatus 400 may include a duct 402 and a head 404.

The duct 402 may facilitate the user to administer the inserting apparatus 400 along the gastrointestinal tract 302 of the subject.

The head 402 may be coupled with the duct 402.

The head 402 may include a docking region 406 and a tip 408.

The docking region 406 may facilitate the clinician to load the capsular device 100 of FIG. 1 in the insertion apparatus 400. The insertion apparatus 400 of FIG. 4 is not only limited to carry the capsular device 100 but also suitable to carry the pellet device 200.

In some aspects of the present disclosure, the docking region 406 may include a magnet (not shown), that may be adapted to dock and undock the capsular device 100 in the insertion apparatus 400. In some aspects of the present disclosure, the docking region 406 may include a fastener (not shown), that may be adapted to dock and undock the capsular device 100 in the insertion apparatus 400.

The tip 408 may be positioned on a distal side of the insertion apparatus 400 (for purposes of this disclosure, “distal side” refers to the portion of the insertion apparatus 400 that is far from the user during normal use). The tip 408 may facilitate the insertion apparatus 400 to penetrate into the contracted muscles of the gastrointestinal tract 302.

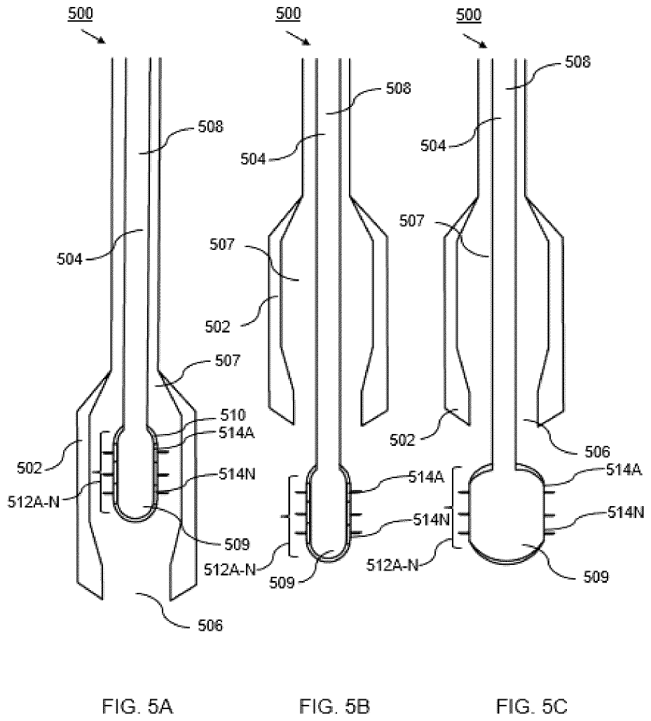

FIGS. 5A, 5B, and 5C illustrates an installing apparatus 500, in accordance with an aspect of the present disclosure.

The installing apparatus 500 may include a core 502 and an inflation equipment 504.

The core 502 may facilitate the clinician to administer the installing apparatus 500 along the gastrointestinal tract 302 of the subject.

The inflation equipment 504 may be positioned in the core 502 of the installing apparatus 500.

The inflation equipment 504 may include a pipe 508 and a bladder 509.

The pipe 508 may be operatively positioned inside the core 502 of the insertion apparatus. The pipe 508 may be adapted to move in a substantially vertical direction along an axis of the inflation equipment 504.

The bladder 509 may be coupled with the pipe 508 and adapted to inflate, upon receiving the pumping agent from the tube 508. The pipe 508 may be adapted to push the bladder 509 from the core 502 through a bore 508 provided on a distal end of the core 502.

The bladder may further include an expandable layer 510, one or more nails 512A-512N, and one or more compartments 514A-514N.

The expandable layer 510 may be adapted to inflate upon receiving the pumping agent from the pipe 508.

Each nail of the one or more nails 512A-512N may be mounted over the expandable layer 510. Upon inflating, each nail of the one or more nails 512A-512N may be adapted to inject one or more samples into the region of interest 304 of the subject. The utilities of the one or more nails 512A-512N are substantially similar to the utilities of the one or more needles 108A-108N of capsular device 100. In order to differentiate the components with respect to Figures alike terms are used.

At least one compartment of the one or more compartments 514A-514N may be positioned on proximal end of each nail of the one or more nails 512A-512N.

Each compartment of the one or more compartments 514A-514N may be adapted to store at least one sample of the one or more samples.

In some aspects of the present disclosure, each compartment of the one or more compartments 514A-154N may include at least one offline RFID sensor. In some other aspects of the present disclosure, the RFID sensor facilitates the clinician to locate the regions of interest where each nail of the one or more nails 512A-512N is injected.

Upon inflating, the pumping agent squeezes each compart of the one or more compartments 512A-512N. Such that at least one sample of the one or more samples stored in each compartment of the one or more compartments 514A-514N is injected into the region of interest 304 of the subject by way of the one or more nails 512A-512N.

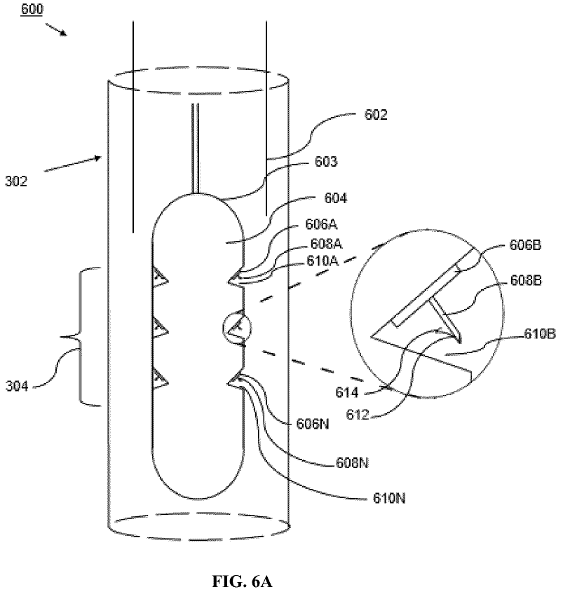

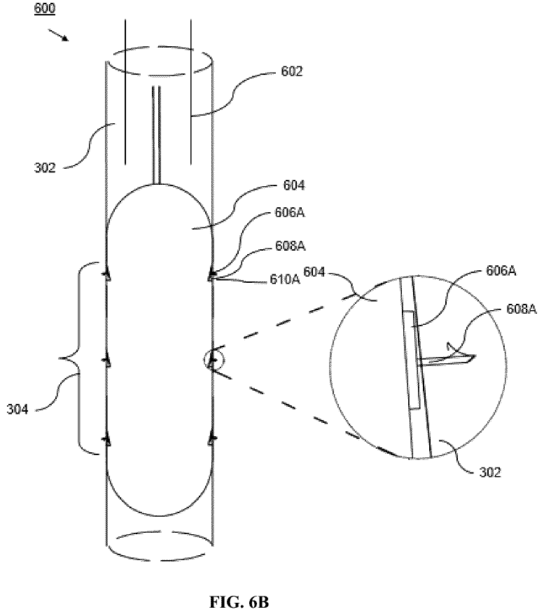

FIGS. 6A and 6B illustrates an implanting apparatus 600, in accordance with an aspect of the present disclosure.

The implanting apparatus 600 may include a delivery device 602 and an inflation equipment 603.

The delivery device 602 may facilitate the clinician to administer the implanting apparatus 600 along a gastrointestinal tract 302 of the subject.

The inflation equipment 603 may be operatively positioned inside the delivery device 602 of the implanting apparatus 600.

The inflation equipment 603 may include a bag 604.

The bag 604 may be adapted to inflate, upon receiving the pumping agent provided by the clinician.

The bag 604 may include one or more chambers 606A-606N, one or more infusers 608A-608N, and one or more cavities 610A-610N.

Each cavity of the one or more cavities 610A-610N may be positioned on the bladder 604 and adapted to project externally upon inflation.

At least one chamber of the one or more chambers 606A-606N may be positioned on each cavity of the one or more cavities 610A-610N.

Each chamber of the one or more chambers 606A-606N may be adapted to store at least one sample of one or more samples.

At least one infuser of the one or more infusers 608A-608N may be positioned on each chamber of the one or more chambers 606A-606N. The utilities of the one or more infusers 608A-608N are substantially similar to the utilities of the one or more needles 108A-108N of capsular device 100. In order to differentiate the components with respect to Figures alike terms are used.

Each infuser of the one or more infusers 608A-608N may be adapted to transmit corresponding sample of the one or more samples stored in a respective chamber of the one or more chambers 606A-606N to the regions of interest 304 of the subject. Each infuser of the one or more infusers 608A-608N may include a hook 612.

The hook 612 may be positioned on a distal end of each infuser of the one or more infusers 608A-608N.

Each infuser of the one or more infusers 608A-608N may be adapted to bind with the region of interest 304 of the gastrointestinal tract 302. In some aspects of the present disclosure, the hook 612 may be adapted to bind the region hook 612 in the region of interest 304.

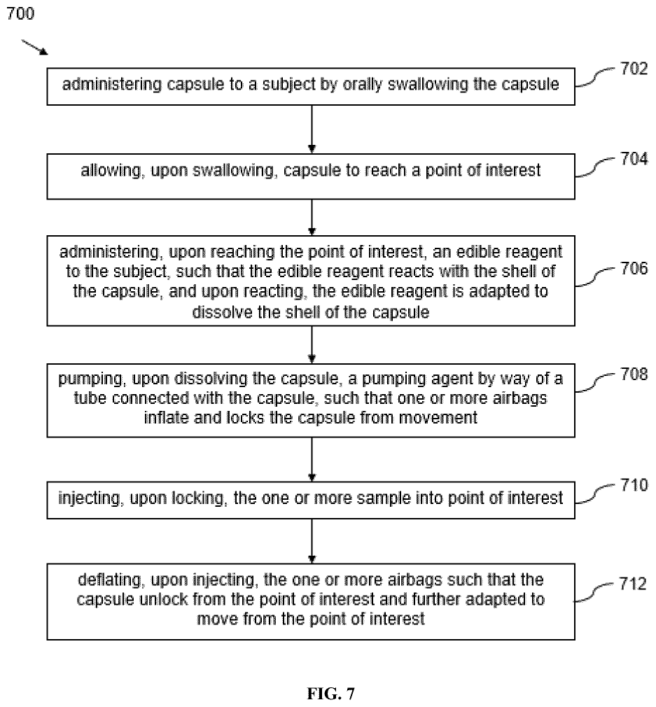

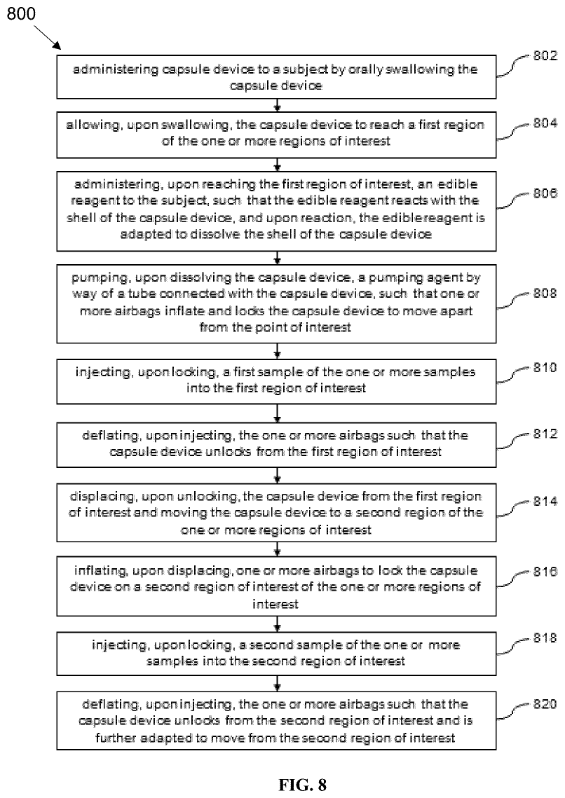

FIG. 7 illustrates a flowchart that depicts a method 700 of administering one or more samples to the point of interest of the subject, in accordance with an aspect of the present disclosure. The method 700 may include the following steps:

At step 702, administering the capsular device 100 to the subject by orally swallowing the capsular device 100.

At step 704, allowing, upon swallowing, the capsular device 100 to reach a point of interest.

At step 706, administering, upon reaching the point of interest, an edible solution to the subject, such that the edible solution reacts with the shell of the capsular device 100, and upon reaction, the edible reagent is adapted to dissolve the shell 104 of the capsular device 100. In some aspects of the present disclosure, upon administering the capsular device, the capsular device 100 may be stopped at the point of interest. In some aspects of the present disclosure, the capsular device 100 may be stopped manually by administering a sticking solution.

At step 708, pumping, upon dissolving the capsular device, the pumping agent by way of the tube 102 coupled with the capsular device 100, such that each airbag of the one or more airbags may inflate, locks the capsular device 100, and prevents the capsular device 100 to move apart from the point of interest.

At step 710, injecting, upon locking, the one or more samples into the point of interest.

At step 712, deflating, upon injecting, each airbag of the one or more airbags 106A-106N, such that the capsule unlocks from the point of interest and is further adapted to move from the point of interest.

At step 714, quantifying and analysing an elicited immune response in the point of interest of the subject at regular intervals.

At step 716, formulating a personalized diet plan for the subject based on the analyzed data obtained from the step 714.

FIG. 8 illustrates a flowchart that depicts a method 800 of administering one or more samples to one or more regions of interest of the subject, in accordance with an aspect of the present disclosure. The method 800 may include the following steps:

At step 802, administering the capsular device 100 to the subject by orally swallowing the capsular device 100.

At step 804, allowing, upon swallowing, the capsular device to reach a first region of the one or more regions of interest.

At step 806, administering, upon reaching the first region of interest, an edible reagent to the subject, such that the edible reagent reacts with the shell 104 of the capsular device 100, and upon reaction, the edible reagent is adapted to dissolve the shell 104 of the capsular device 100.

At step 808, pumping, upon dissolving the capsular device 100, the pumping agent by way of a tube 102 connected with the capsular device 100, such that one or more airbags 106A-106N inflate and locks the capsular device 100 to move apart from the point of interest.

At step 810, injecting, upon locking, a first sample of the one or more samples into the first region of interest.

At step 812, deflating, upon injecting, the one or more airbags 106A-106N such that the capsular device 100 unlocks from the first region of interest.

At step 814, displacing, upon unlocking, the capsular device 100 from the first region of interest and moving the capsular device 100 to a second region of the one or more regions of interest.

At step 816, inflating, upon displacing, one or more airbags 106A-106N to lock the capsular device 100 on a second region of interest of the one or more regions of interest. At step 818, injecting, upon locking, a second sample of the one or more samples into the second region of interest.

At step 820, deflating, upon injecting, the one or more airbags 106A-106N such that the capsular device 100 unlocks from the second region of interest and may further be adapted to move from the second region of interest.

At step 822, quantifying and analyzing an elicited immune response in the one or more regions of interest of the subject at regular intervals.

At step 824, formulating a personalized diet plan for the subject based on the analyzed data obtained from the step 822.

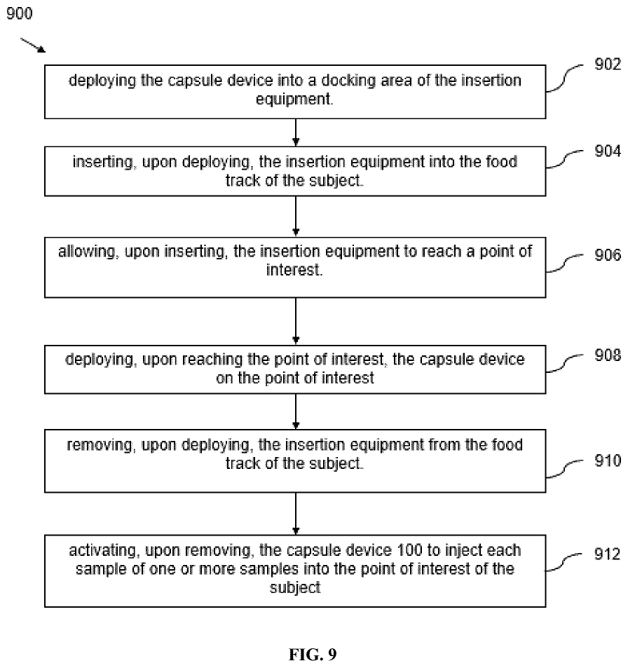

FIG. 9 illustrates a method 900 of administering the capsular device 100 to a point of interest of the subject by way of the insertion equipment 900, in accordance with an aspect of the present disclosure. The method 900 may include the following steps: At step 902, deploying the capsular device 100 into a docking region 406 of the insertion equipment 400.

At step 904, inserting, upon deploying, the insertion equipment 400 into the gastrointestinal tract of the subject.

At step 906, allowing, upon inserting, the insertion equipment 400 to reach a point of interest.

At step 908, deploying, upon reaching the point of interest, the capsular device 100 on the point of interest.

At step 910, removing, upon deploying, the insertion equipment 400 from the gastrointestinal tract 302 of the subject.

At step 912, activating, upon removing, the capsular device 100 to inject each sample of one or more samples into the point of interest of the subject.

In some aspects of the present disclosure, the point of interest may be in one or more regions of a gastrointestinal wall of the subject. In some aspects of the present disclosure, the point of interest may be in one or more regions of an esophageal wall of the subject.

In some aspects of the present disclosure, the subject may be one of, but not limited to, a human or an animal.

In some aspects of the present disclosure, the one or more regions of interest may include, but not limited to, wound, muscle, bone, artery, vein, bone marrow, nasal passage, food passage, esophagus, oral cavity, stomach, small intestine, and large intestine.

In some aspects of the present disclosure, the one or more samples may be injected by way of an endoscopic or a non-endoscopic method. In some aspects of the present disclosure, the one or more samples may be one or more allergens. In some aspects of the present disclosure, the one or more allergens may be one of, soy, milk, peanuts, sesame, wheat, fish, shellfish, tree nuts, dairy products, and the like.

In some aspects of the present disclosure, the immune response of the subject may be quantified and analyzed by a technician, a doctor, a researcher, a research scholar, software, an artificial intelligence system, a symptom monitoring device, by collecting tissues, visual inspection, capsule endoscope with a string attached, thermal imaging, spectroscopic catheter-based imaging, radiological contrast, non-contrast-enhanced imaging and the like. Aspects of the present disclosure are intended to include or otherwise cover any type of analysis including known, related art, and/or later developed analysis or analysis device.

In some aspects of the present disclosure, one or more sensors may be one of, a temperature sensor, power of Hydrogen (pH) sensor, breath monitor, infection detector, bleeding detector, microorganism detector, imaging sensor, or texture detector.

In some aspects of the present disclosure, the one or more parameters are one of, temperature, power of Hydrogen (pH), breath level, microbial infection, or bleeding.

In some aspects of the present disclosure, the results may be quantified and analyzed by a doctor, a nurse, a technician, a researcher, a research scholar, a nutritionist, a healthcare provider or a trained personnel.

In some aspects of the present disclosure, the diet plan may be formulated by a doctor, a nurse, a nutritionist, a healthcare provider, or trained personnel.

In some aspects of the present disclosure, the diet plan is formulated by one of, but not limited to, a software, an artificial intelligence, a researcher, or a research scholar.

In some aspects of the present disclosure, the formulated diet plans may be optionally stored in a storage device (not shown). The storage device may facilitate the doctor to update periodically and modify the food diet if required.

In some aspects of the present disclosure, the one or more samples may introduce into the skin to observe one or more allergic reactions in the skin.

In some aspects of the present disclosure, the one or more samples may be replaced with one or more therapeutic agents, such that the capsular device 100 or the pellet device 200 may facilitate to deliver of each therapeutic agent of the one or more therapeutic agents to a respective region of the one or more regions of interest.

In an exemplary scenario, when the subject is injected with the one or more samples by way of the capsular device 100 or the pellet device 200. The clinician may observe the reaction of the one or more samples in each region of the one or more regions of interest. Upon observing the reaction in each region of the one or more regions of interest, the clinician may inject one or more therapeutic agents into respective regions in the one or more regions of interest.

In some aspects of the present disclosure, the one or more needles may be a dissolvable needle. Upon injecting the one or more samples, the one or more needles may dissolve in the body fluids.

In some aspects of the present disclosure, the one or more needles may be a nano-needle.

The foregoing discussion of the present disclosure has been presented for purposes of illustration and description. It is not intended to limit the present disclosure to the form or forms disclosed herein. In the foregoing Detailed Description, for example, various features of the present disclosure are grouped in one or more aspects, configurations, or aspects for the purpose of streamlining the disclosure. The features of the aspects, configurations, or aspects may be combined in alternate aspects, configurations, or aspects other than those discussed above. This device of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights which include alternative aspects, configurations, or aspects to the extent permitted, including alternate, interchangeable, and/or equivalent structures, functions, ranges, or steps to those claimed, whether or not such alternate, interchangeable and/or equivalent structures, functions, ranges or steps are disclosed herein, and without intending to publicly dedicate any patentable subject matter.

We claim:

1. A capsular device (100) comprising:
one or more airbags (106A-106N), wherein each airbag of the one or more airbags (106A-106N) is positioned on both proximal and distal sides of the capsular device (100), and wherein the one or more airbags (106A-106N) are adapted to inflate and lock the capsular device (100) in a point of interest in a gastrointestinal tract and prevents the capsular device (100) to move from the point of interest; and
one or more needles (108A-108N) are positioned in the capsular device (100), and each needle of the one or more needles (108A-108N) is adapted to move in a substantially horizontal direction and adapted to inject at least one sample of one or more samples to the point of interest on the gastrointestinal tract.

2. The capsular device (100) as claimed in claim 1, further comprising a tube (102), such that the tube (102) facilitates a clinician to pump a pumping agent into each airbag of the one or more airbags (106A-106N).

3. The capsular device (100) as claimed in claim 2, wherein the tube (102) facilitates the clinician to dispense each sample of one or more samples to the points of interest.

4. The capsular device (100) as claimed in claim 1, wherein each needle of the one or more needles (108A-108N) comprises at least one payload of one or more payloads (112A-112N), each payload of the one or more payloads (112A-112N) is adapted to store at least one sample, upon injection, each payload of the one or more payloads (112A-112N) is adapted to provide a corresponding sample of the one or more samples to a corresponding needle of the one or more needles (108A-108N).

5. The capsular device (100) as claimed in claim 1, further comprising a control unit (110) that is positioned inside the capsular device (100), adapted to control a flow of samples to the respective needle of the one or more needles (108A-108N).

6. The capsular device (100) as claimed in claim 5, wherein the control unit (108) further comprises a Radio Frequency Identity Card (RFID), that facilitates a clinician to locate the capsular device (100) in a subject.

7. The capsular device (100) as claimed in claim 5, wherein the control unit (108) further comprises one or more sensors, that are configured to detect one or more parameters associated with a subject.

8. A capsular device (100) comprising:
one or more airbags (106A-106N), wherein each airbag of the one or more airbags (106A-106N) is positioned on both proximal and distal sides of the capsular device (100), and wherein the one or more airbags (106A-106N) are adapted to inflate and lock the capsular device (100) in a point of interest in a gastrointestinal tract and prevents the capsular device (100) to move from the point of interest; and
one or more needles (108A-108N) are positioned in the capsular device (100), and each needle of the one or more needles (108A-108N) is adapted to move in a substantially horizontal direction and adapted to inject at least one allergen of one or more allergens to the point of interest in the gastrointestinal tract.

9. The capsular device (100) as claimed in claim 8, wherein each needle of the one or more needles (108A-108N) comprises at least one payload of one or more payloads (112A-112N) such that the at least one payload of the one or more payloads (112A-112N) stores (i) the at least one allergen and (ii) one or more agents, wherein the one or more agents is one of, food grade dyes and coloring agents, wherein the at least one allergen is mixed with the one or more agents in the at least one payload of the one or more payloads (112A-112N).

10. The capsular device (100) as claimed in claim 8, wherein the point of interest is mucosa in the gastrointestinal tract.

11. A pellet device (200) comprising:
a housing (204) adapted to encapsulate the pellet device (200);
a pair of locking bags (206A-206B), wherein at least one locking bag of the pair of locking bags (206A-206B) is positioned on both proximal and distal end of the pellet device (200), adapted to lock the pellet device (200) in a region of interest (304) in a gastrointestinal tract (302), and prevent the pellet device (200) to move from the region of interest (304);
one or more cameras (208A), each camera of the one or more cameras (208A-208N) is positioned on the pellet device (200), facilitates a clinician to visualize walls of the gastrointestinal tract (302), such that the clinician identifies the region of interest (304);
a suction pump (210), that is adapted to create vacuum in a region (308) between the region of interest (304) and the pellet device (200); and
one or more pins (212A-212N), each pin of the one or more pins (212A-212N) is adapted to move in a substantially horizontal direction and inject at least one sample of one or more samples to the region of interest (304).

12. The pellet device (200) as claimed in claim 11, further comprising a hose (202), that is positioned on the pellet device (200) and facilitates the clinician to operate the pellet device (200).

13. The pellet device (200) as claimed in claim 11, further comprising one or more detectors (214), that are adapted to detect one or more parameters.

14. An installing apparatus (500) comprising:
a core (502) adapted to be administered along a gastrointestinal tract of a subject;
an inflation equipment (504) comprising:
a pipe (508) that is operatively positioned inside the core (502) of the installing apparatus (500); and
a bladder (509) that is coupled with the pipe (508), the bladder (508) comprising:
an expandable layer (510) that is adapted to inflate, upon receiving a pumping agent from the pipe (508);
one or more nails (512A-512N), each nail of the one or more nails (512A-512N) is mounted over the expandable layer (510), and upon inflating, each nail of the one or more nails (512A-512N) is adapted to inject one or more samples into a region of interest (304) of a subject;
one or more compartments (514A-514N), at least one compartment of the one or more compartments (514A-514N) is positioned on proximal end of each nail of the one or more nails (512A-512N), and each compartment of the one or more compartments (514A-514N) is adapted to store at least one sample of the one or more sample, upon inflating the pumping agent squeezes each compart of the one or more compartments (512A-512N), such that at least one sample of the one or more samples stored in each compartment of the one or more compartments (514A-514N) is injected to the region of interest (304) of the subject by way of the one or more nails (512A-512N).

15. An implanting apparatus (600) comprising:
a delivery device (602) administered along a gastrointestinal tract (302) of a subject;
an inflation equipment (603) that is operatively positioned inside the delivery device (602) of the implanting apparatus (600), the inflation equipment (603) comprising:
a bag (604) comprising:
one or more cavities (610A-610N) that are positioned on the bladder (604) and adapted to project externally upon inflation;
one or more chambers (606A-606N), wherein at least one chamber of the one or more chambers (606A-606N) is positioned on each cavity of the one or more cavities (610A-610N), and wherein each chamber of the one or more chambers (606A-606N) is adapted to store at least one sample of one or more samples; and
one or more infusers (608A-608N), wherein at least one infuser of the one or more infusers (608A-608N) is positioned on each chamber of the one or more chambers (606A-606N), and wherein the one or more infusers (608A-608N) adapted to transmit corresponding sample of the one or more samples stored in a respective chamber of the one or more chambers (606A-606N); and
wherein each infuser of the one or more infusers (608A-608N) comprising a hook (612), that is positioned on a distal end of each infuser of the one or more infusers (608A-608N) and facilitates the one or more infusers (608A-608N) to bind with a region of interest (304) of the gastrointestinal tract (302).

16. A method (700) of injecting one or more samples to a point of interest of a subject, the method (700) comprising:
administering (702) capsular device (100) to a subject by orally swallowing the capsular device (100);
allowing (704), upon swallowing, the capsular device (100) to reach a point of interest;
administering (706), upon reaching the point of interest, an edible reagent to the subject, such that the edible reagent reacts with the shell (104) of the capsular device (100), and upon reaction, the edible reagent is adapted to dissolve the shell (104) of the capsular device (100);
pumping (708), upon dissolving the capsular device (100), air by way of a tube (100) connected with the capsular device (100), such that one or more airbags (100) inflate and locks the capsular device to move apart from the point of interest;
injecting (710), upon locking, the one or more samples into the point of interest; and
deflating (712), upon injecting, the one or more airbags (100) such that the capsule unlocks from the point of interest and is further adapted to move from the point of interest.

17. A method (800) of injecting one or more samples into one or more regions of interest of the subject, the method (800) comprising:
administering (802) capsular device (100) to a subject by orally swallowing the capsular device (100);
allowing (804), upon swallowing, the capsular device (100) to reach a first region of the one or more regions of interest;
administering (806), upon reaching the first region of interest, an edible reagent to the subject, such that the edible reagent reacts with the shell (104) of the capsular device (100), and upon reaction, the edible reagent is adapted to dissolve the shell (104) of the capsular device (100);
pumping (808), upon dissolving the capsular device (100), air by way of a tube (102) connected with the capsular device (100), such that one or more airbags (106A-106N) inflate and locks the capsular device (100) to move apart from the point of interest;
injecting (810), upon locking, a first sample of the one or more samples into the first region of interest;
deflating (812), upon injecting, the one or more airbags (106A-106N) such that the capsular device (100) unlocks from the first region of interest;
displacing (814), upon unlocking, the capsular device (100) from the first region of interest and moving the capsular device (100) to a second region of the one or more regions of interest;
inflating (816), upon displacing, one or more airbags (106A-106N) to lock the capsular device (100) on a second region of interest of the one or more regions of interest;
injecting (818), upon locking, a second sample of the one or more samples into the second region of interest; and
deflating (820), upon injecting, the one or more airbags (106A-106N) such that the capsular device (100) unlocks from the second region of interest and is further adapted to move from the second region of interest.

18. A method (900) of administering a capsular device (100) to a point of interest of a subject by way of an insertion equipment (400), the method comprising:
deploying (902) the capsular device (100) into a docking region (406) of the insertion equipment (400);
inserting (904), upon deploying, the insertion equipment (400) into a gastrointestinal tract (302) of the subject;
allowing (906), upon inserting, the insertion equipment (400) to reach a point of interest;
deploying (908), upon reaching the point of interest, the capsular device (100) on the point of interest;
removing (910), upon deploying, the insertion apparatus from the gastrointestinal tract (302) of the subject; and
activating (912), upon removing, the capsular device (100) to inject each sample of one or more samples into the point of interest of the subject.
